(19) United States
(12) Patent Application Publication
(10) Pub. No.: US 2005/0136126 A1
(43) Pub. Date: Jun. 23, 2005

(54) Agent for reduction of scar formation by using wound alkalinization

(75) Inventors: Ki Chang Keum, Seoul (KR); Naechoon Yoo, Seoul (KR); Won-min Yoo, Seoul (KR)

Correspondence Address: JHK LAW, P.O. BOX 1078, LA CANADA, CA, 91012-1078, US

(73) Assignee: CambridgeMED, Inc.

(21) Appl. No.: 11/028,767

(22) Filed: Jan. 3, 2005

Related U.S. Application Data
(63) Continuation-in-part of application No. 10/312,097, filed on Mar. 17, 2003.

(51) Int. Cl. A61K 33/42; A61K 33/00

(52) U.S. Cl. 424/601; 424/722; 424/717

(57) ABSTRACT

The present invention relates to an agent for reducing scar formation, which inhibits the scar formation by injecting sodium bicarbonate to the dermis tissue of the wound with a syringe to directly control pH of the wound site, namely, by alkalinizing the wound to inactivate TGF-β. According to the present invention, the agent for reducing scar formation by controlling the wound healing process can be clinically and immediately applied to the treatment of tylosis scar and keloid, etc., and is effective to the treatment of intractable fibrosis diseases. Also, the invention can be widely applied in many ways as an easy tool which can control the action of TGF-β in a living organism.

### CROSS-REFERENCE TO RELATED APPLICATIONS

The present application is a continuation-in-part (CIP) application of U.S. application Ser. No. 10/312,097, filed Mar. 17, 2003, which is incorporated by reference herein in its entirety.

### TECHNICAL FIELD

The present invention relates to an agent for reducing scar formation by inducing alkalinization of a wound site, which is clinically easy to apply. More particularly, the present invention relates to an alkalinizing agent for reducing scar formation by, upon administration to the dermis tissue of a wound site, increasing the pH of a wound site to induce an alkalinization of said wound site, followed by inactivating TGF-β and thereby inhibiting the scar formation on the wound site. In addition, the present invention relates to a method for reducing scar formation on a wound site comprising administering to said wound site an effective amount of an alkalinizing agent to alkalinize said wound site and allowing the wound site to heal thereby reducing scar formation on the healing site. Examples of such an alkalinizing agent include sodium bicarbonate, sodium hydroxide and potassium hydroxide.

### BACKGROUND ART

Many efforts have been made to reduce or inhibit the formation of scar. However, an effective method has not yet been found. There was a report showing that the wound of a fetus was cured without scar. After such a report, trials have been made to reveal the differences between the wound healing process in a fetus and that in a mature individual, and change the wound healing process from in a mature individual to in a fetus as an approach to reduce the scar formation. One of the revealed differences is that transforming growth factor-beta (TGF-β) is not immunochemically detected in the wound of a fetus, but functions in the wound healing process of a mature individual and plays an important role in the scar formation. This fact shows that it would be possible to inhibit the scar formation by controlling TGF-β. Actually, there was a report that the scar formation in a white rat could be reduced by the use of an antibody against TGF-β (Acidic cellular environments; activation of latent TGF-β and sensitization of cellular responses to TGF-β and EGF. International Journal of Cancer. 43(5):886-91, May 15, 1989). TGF-β is secreted in inactivated state from cells and is mostly present in inactivated state even outside of cells. Only when exposed to a certain specific condition, TGF-β is activated, combines with a receptor of a target cell and functions. Heat, acidic condition, protease, etc. are known as physicochemical conditions which can activate TGF-β (Physicochemical activation of recombinant latent transforming growth factor-beta's 1, 2, and 3. Growth Factors. 3(1):35-43, 1990). However, no studies have been made yet with respect to the reduction of scar formation by using the inactivation of TGF-β. The present invention aims to control the pH of a wound site, which is clinically easy to apply, namely, to alkalinize said wound site and lead the inactivation of TGF-β, and to confirm whether the inactivation actually reduces the scar formation.

There have been several trials to reduce scar formation by controlling TGF-β which functions during the entire process of scar formation. However, those trials were related to the use of an antibody and have problems in actual application. TGF-β widely functions in a living organism and is under active study in various fields. The processes of formation, secretion and function of TGF-β have not yet been clearly known. According to the facts discovered to date, TGF-β is secreted from cells in inactivated state and functions with a target organ under specific conditions. Heat, acidic pH, and protease are known as the physicochemical conditions which can activate TGF-β. However, no trials have been made to reduce the scar using TGF-β.

### DISCLOSURE OF INVENTION

The present invention was conceived based upon the above stated facts. The inventors of the present invention have completed the present invention by controlling the pH of the wound, which is clinically easy to apply, that is, by alkalinizing the wound and inactivating TGF-β, thereby leading to the wound healing process which is similar to that of a fetus and confirming that the scar formation is reduced. Accordingly, the objective of the present invention is to provide an agent for reducing scar formation, which inactivates TGF-β, reduces the scar formation and thus heals the wound without scar.

The present invention is directed to a method for reducing scar formation during the wound healing process. TGF-β is known to play an important role in the scar formation process of the wound. To inactivate TGF-β, the wound should be alkalinized. To easily alkalinize the wound, in one exemplification of the invention, an alkalinizing agent such as sodium bicarbonate, NaOH or KOH in a concentration of 1 mEq (1 cc)/distilled water 10 cc is injected to the dermis tissue of the wound to directly control pH of the wound, thereby reducing the scar formation of the wound. The pH range which effectively reduces the scar formation, namely, which does not activate TGF-β and alkalinizes the wound, may be 8.5-10.0, preferably 9.0-10.0, more preferably 9.2-9.8 for optimal performance. Also, alkalinizing agents of the present invention such as sodium bicarbonate, NaOH and KOH reduce fibrosis in the process of tissue regeneration or healing, by controlling pH (alkalinization) of the regeneration or healing spot. Thus, stated in another way, the present invention is directed to a method for reducing scar formation at a wound site comprising administering to the wound site an effective amount of an alkalinizing agent to raise pH at the wound site so as to inactivate TGF-β at the wound site and allowing the wound site to heal thereby reducing scar formation at the healing site. The alkalinizing agent may be injected into the dermis tissue of the wound site. In particular, the alkalinizing agent may be sodium bicarbonate, sodium hydroxide, potassium hydroxide, tromethamine (THAM), acetazolamide (Diamox), sodium citrate, potassium citrate, phosphate salts or sodium lactate.

The present invention is also directed to a pharmaceutical composition for reducing scar formation at a wound site comprising an effective amount of an alkalinizing agent to raise pH at the wound site and inactivate TGF-β at the wound site and a pharmaceutically acceptable carrier. The raised pH at the wound site may be in the range of 8.5 to 10.0 or 9.2 to 9.8 for optimal performance. The composition may be in a form of an injection solution, and the alkalinizing agent may be present in a concentration of about 0.1 to 1 mEq/0. 1 cc of purified water. In particular, the alkalinizing agent may be sodium bicarbonate, sodium hydroxide, potassium hydroxide, tromethamine (THAM), acetazolamide (Diamox), sodium citrate, potassium citrate, phosphate salts (potassium phosphate, sodium acid phosphate, and tribasic sodium phosphate) or sodium lactate.

The present invention also encompasses a kit for reducing scar formation at a wound site comprising: (i) at least one container containing an effective amount of an alkalinizing agent to raise pH at the wound site and inactivate TGF-β at the wound site and a pharmaceutically acceptable carrier, wherein the raised pH at the wound site may be in the range of 8.5 to 10.0 or 9.2 to 9.8 for optimal performance. The composition may be in a form of an injection solution, and the alkalinizing agent may be present in a concentration of about 0.1 to 1 mEq/0.1 cc of purified water, in particular, the alkalinizing agent may be sodium bicarbonate, sodium hydroxide, potassium hydroxide, tromethamine (THAM), acetazolamide (Diamox), sodium citrate, potassium citrate, phosphate salts (potassium phosphate, sodium acid phosphate, and tribasic sodium phosphate) or sodium lactate; and (ii) written instructions on the use of the kit for reducing scar formation.

Regarding concentrations and amounts of the various reagents, please refer to Tables 1 and 2 below:

TABLE 1
(pH value)

| | | 0.1 | 0.5 | 1.0 |
|---|---|---|---|---|
| | 0.05 mEq | mEq | mEq | mEq |
| NaHCO₃ | 8.83 | 9.22 | 9.82 | 10.36 |
| KOH | 8.56 | 9.40 | 9.74 | 10.51 |
| NaOH | 8.55 | 9.38 | 9.87 | 10.50 |

TABLE 2
(mg per 10 ml of distilled water)

| | 0.05 mEq | 0.1 mEq | 0.2 mEq | 0.5 mEq | 1.0 mEq |
|---|---|---|---|---|---|
| NaHCO₃ | 0.020 | 0.040 | 0.080 | 0.200 | 0.400 |
| KOH | 0.028 | 0.056 | 0.112 | 0.280 | 0.560 |
| NaOH | 0.042 | 0.084 | 0.168 | 0.420 | 0.840 |

Tables 1 and 2 make it possible for those skilled in the art to determine concentration and amount of each reagent in inducing the alkaline pHs 8.5-10 at a wound site.

The present composition can be administered to a wound site by any appropriate route, for example, parenterally, intradermally, subcutaneously, intramuscularly or topically, in forms of solution, suspension, cream, lotion, ointment, gel and the like. Solutions or suspensions used for parenteral, intradermal, subcutaneous, intramuscular or topical application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminctetraacetic acid and agents for the adjustment of tonicity such as sodium chloride or dextrose. The parental preparation can be enclosed in ampoules, disposable syringes or multiple dose vials made of glass or plastic. For application topically to the wound site, the instant composition can be formulated as a suitable ointment containing the alkalinizing agent suspended or dissolved in, for example, a mixture with one or more of the following: mineral oil, liquid petrolatum, white petrolatum, propylene glycol, polyoxyethylene polyoxypropylene compound, emulsifying wax and water. For a suitable lotion or cream, the alkalinizing agent can be suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water. A typical topical dose of the alkalinizing agent will range from 0.1-4.0 % wt/wt in a suitable carrier.

### BRIEF DESCRIPTION OF THE DRAWINGS

FIG. 1a shows the tissue of the control group after two weeks from the injection of distilled water, instead of sodium bicarbonate (dyeing: hematoxylin-eosin dyeing, magnification: 40×).

FIG. 1b shows the tissue of the test group after two weeks from the injection of sodium bicarbonate (dyeing: hematoxylin-eosin dyeing, magnification: 40×).

FIG. 2a shows the tissue of the control group after two weeks from the injection of distilled water, instead of sodium bicarbonate (dyeing: trichrome dyeing, magnification: 400×).

FIG. 2b shows the tissue of the test group after two weeks from the injection of sodium bicarbonate (dyeing: trichrome dyeing, magnification: 400×).

FIG. 3a shows the tissue of the control group after eight weeks from the injection of distilled water, instead of sodium bicarbonate (dyeing: hematoxylin-eosin dyeing, magnification: 40×).

FIG. 3b shows the tissue of the test group after eight weeks from the injection of sodium bicarbonate (dyeing: hematoxylin-eosin dyeing, magnification: 40×).

FIG. 4a shows the tissue of the control group after eight weeks from the injection of distilled water, instead of sodium bicarbonate (dyeing: trichrome dyeing, magnification: 400×).

FIG. 4b shows the tissue of the test group after eight weeks from the injection of sodium bicarbonate (dyeing: trichrome dyeing, magnification: 400×).

FIG. 5a shows the tissue of the test group after two weeks from the injection of TGF-β (dyeing: hematoxylin-eosin dyeing, magnification: 40×).

FIG. 5b shows the tissue of the test group after two weeks from the injection of both TGF-β and sodium bicarbonate (dyeing: hematoxylin-eosin dyeing, magnification: 40×).

FIG. 6a shows the tissue of the test group after eight weeks from the injection of TGF-β (dyeing: hematoxylin-eosin dyeing, magnification: 100×).

FIG. 6b shows the tissue of the test group after eight weeks from the injection of both TGF-β and sodium bicarbonate (dyeing: hematoxylin-eosin dyeing, magnification: 100×).

FIG. 7a shows the tissue of the test group after eight weeks from the injection of TGF-β (dyeing: trichrome dyeing, magnification: 400×).

FIG. 7b shows the tissue of the test group after eight weeks from the injection of both TGF-β and sodium bicarbonate (dyeing: trichrome dyeing, magnification: 400×).

### EXAMPLES

The present invention will be more specifically illustrated by the following examples. The following examples are provided to illustrate the present invention, but are not intended to be limited.

### Example 1

In the wound healing process of an adult white rat, it was histologically studied whether the alkalinization of the wound reduces the scar formation, and it was indirectly confirmed whether such a reduction is caused by TGF-β inactivation, by observing differences of scar formation depending on the action of TGF-β between the alkalinized wound and the control wound.

Materials

Animal: forty white rats of Sprague-Dawley origin, which weigh 300-350 mg

bovine TGF-β
0.1 mEq sodium bicarbonate
   Methods of Experiment
   A. Experiment of Wound Alkalinization (Twenty White Rats)

On the dorsum of adult white rats, skin near the limbs which are on the same distance from the median line was incised in about 10 mm long. In a sham control group, 0.1 cc distilled water was injected to the dermis of the incised wound and simply sutured. In a test group, 0.1 mEq sodium bicarbonate 0.1 cc (pH 9.5±0.3) was injected to the dermis of the incised wound.
a. control×1 site: suture only
b. experimental×1 site: 0.1 mEq sodium bicarbonate 0.1 cc injection and suture
   B. Experiment of TGF-β Inactivation (Twenty White Rats)

On the dorsum of adult white rats, skin near the limbs which are on the same distance from the median line was incised in about 10 mm long. Before suture, TGF-β 0.05 μg was injected to the dermis of the incised wound in test group I, while both TGF-β 0.05 μg and 0.1 mEq sodium bicarbonate 0.1 cc (pH 9.5±0.3) were injected to the dermis of the incised wound in test group II.
a. experimental I×1site: TGF-β injection and suture
b. experimental II×1 sites: TGF-β+sodium bicarbonate injection and suture
   Analysis of the Results
   A. Histological Observation

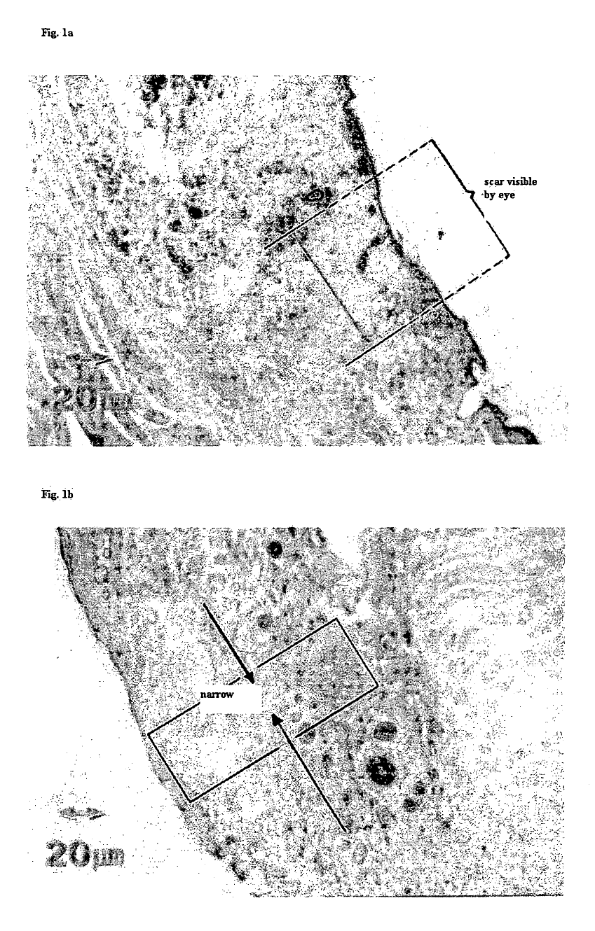

On 14^{th} and 56^{th} days from the experiments, tissues of ten adult white rats were taken, fixed in 10% neutral buffer formalin for 24 hours, embedded in paraffin throughout the normal tissue treating process. In the middle of the wound, the tissue was cut into 4 μm thick, perpendicularly to the major axis, and dyed with hematoxyline-eosine and Masson's trichrome to observe the connective tissues (FIGS. 1a to 7b).

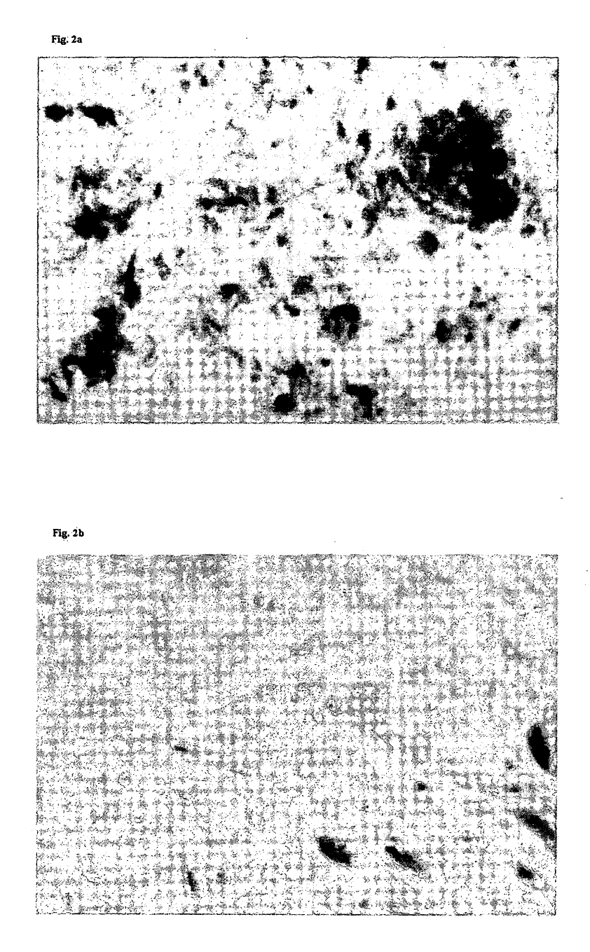

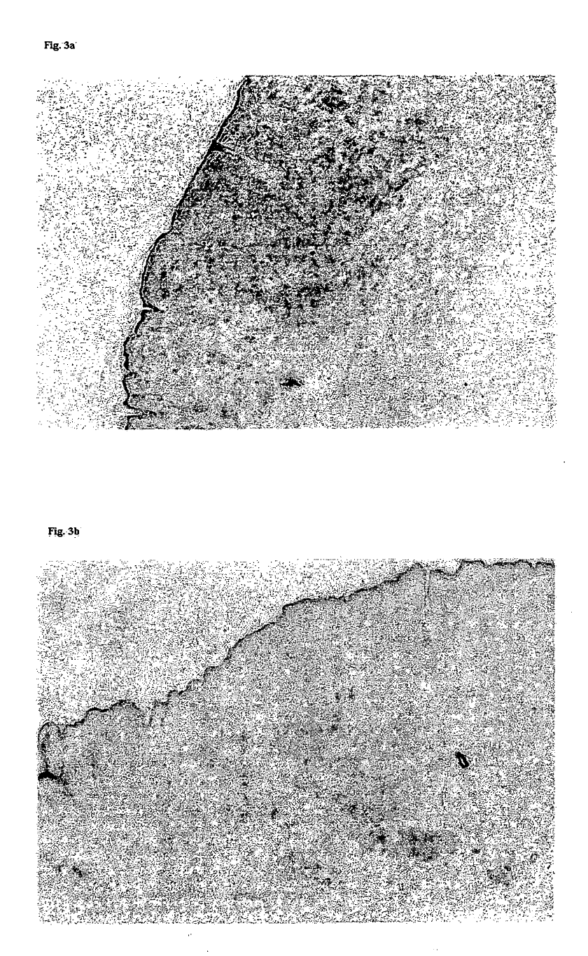

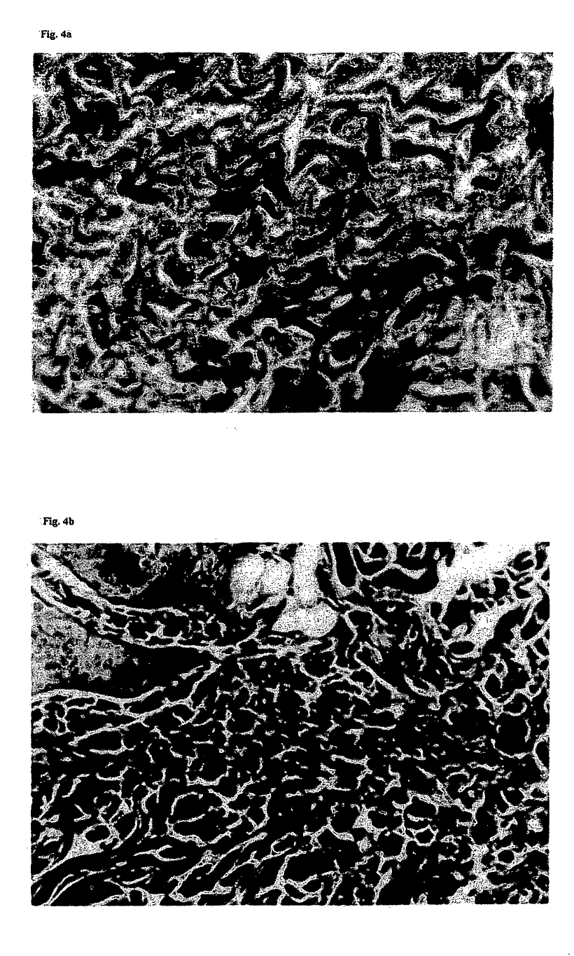

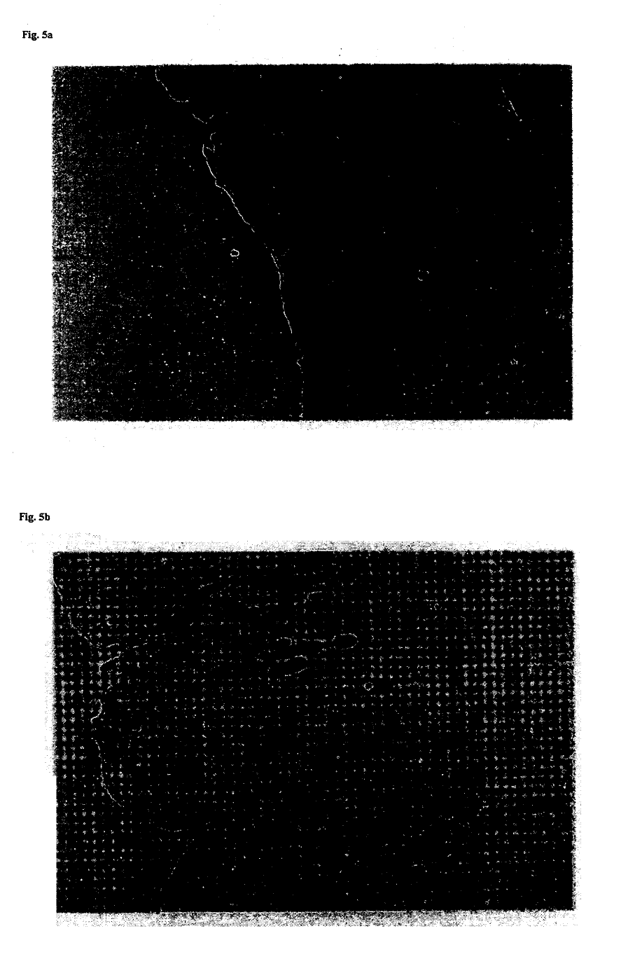

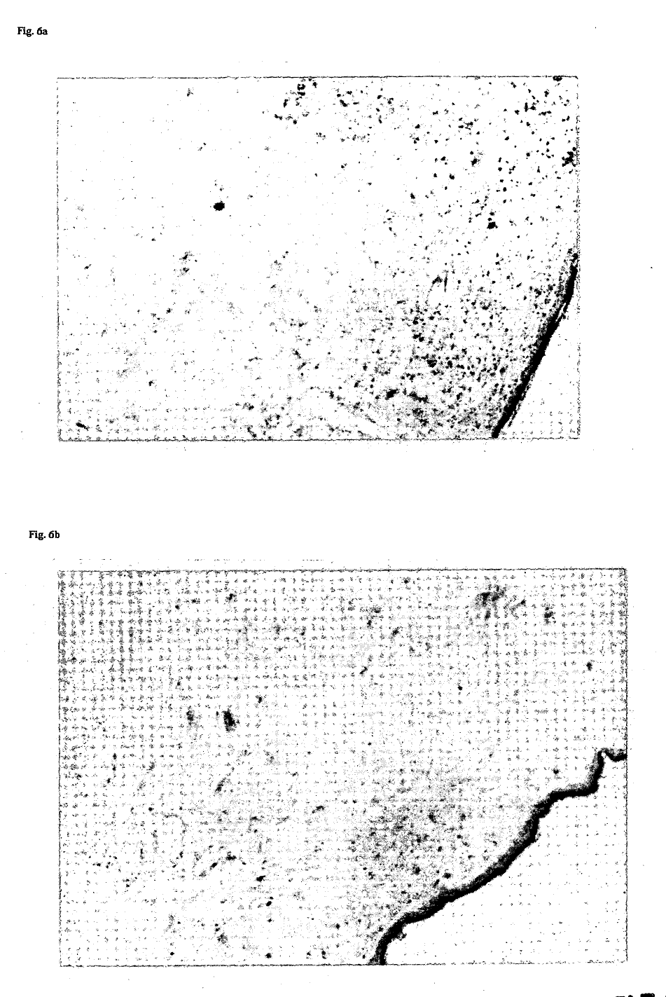

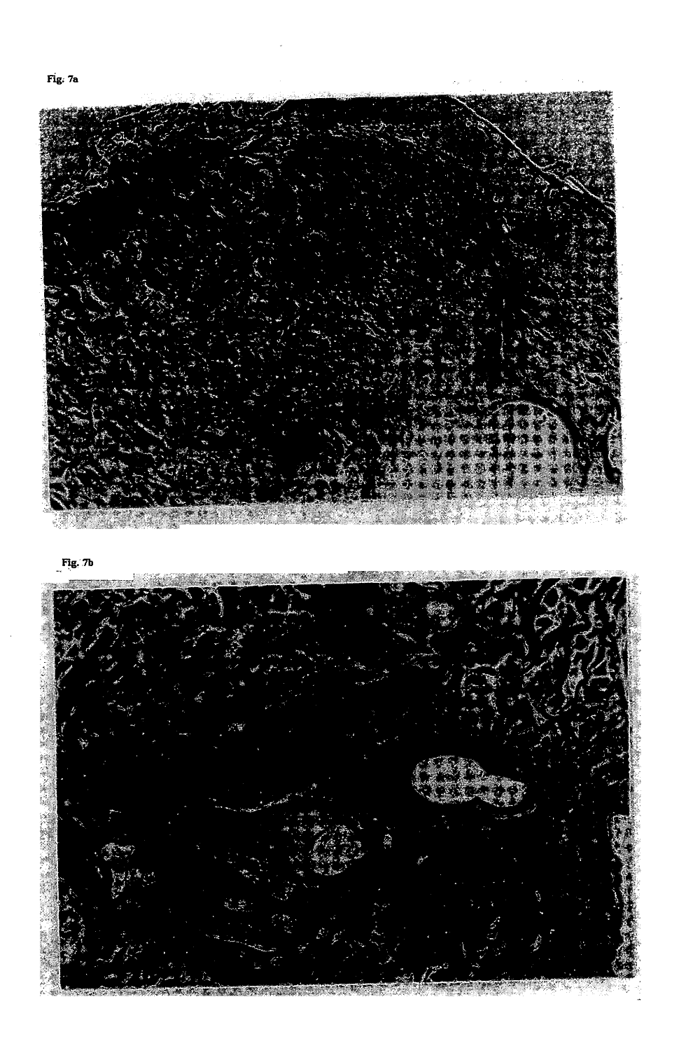

In FIGS. 1a and 1b, control group (a) shows a thicker scar, which is marked with an arrow, than the test group (b). In FIGS. 2a and 2b, control group (a) shows thin, compact, irregular blue-dyed collagen fibers, which are typical collagen appearance of scar. On the other hand, test group (b) shows relatively thick, regular collagen fibers, which are relatively spacious compared to the control group (a). In FIGS. 3a and 3b, control group (a) shows a thicker scar, which is marked with an arrow, than the test group (b). In FIGS. 4a and 4b, control group (a) shows thin, irregular blue-dyed collagen fibers, which are typical collagen appearance of scar. On the other hand, test group (b) shows relatively thick, regular collagen fibers, of which structures are almost similar to the normal dermis nearby. In FIGS. 5a and 5b, TGF-β injected group (a) shows a thicker scar, which is marked with an arrow, than the test group (b). In FIGS. 6a and 6b, TGF-β injected group (a) shows a thicker scar, which is marked with an arrow, than TGF-β+sodium bicarbonate injected group (b). The scar of TGF-β+sodium bicarbonate injected group (b) is too thin to find, and the structure of the tissue is similar to the normal dermis nearby. In FIGS. 7a and 7b, TGF-β injected group (a) shows thin, irregular blue-dyed collagen fibers, which are typical collagen appearance of scar, and forms a thick scar. On the other hand, TGF-β+sodium bicarbonate injected group (b) shows very thin scar and its collagen fibers are thick, regular and of which structure is almost similar to the collagen structure of normal dermis nearby.

B. Measurement of the Width of Scar

With a tissue sample, which was cut perpendicularly to the middle of the wound, the width of the scar by use of image analysis program (Image-Pro version 3.0, Microsoft) under 40 magnification was measured, and the differences between the groups were compared. The result of the measurement was confirmed by Student t-test.

Results

The tissues to be tested were obtained from tested forty white rats, twenty of which were sacrificed at each time, after 2 and 8 weeks from the operational handling.

A. Experiment of Wound Alkalinization

Two (2) weeks after a wound was made, an inflammation was observed from the epidermal tissue layer to the muscular tissue layer at the low magnification (×40) in hematoxylin-eosin dyeing, and there was not much difference between the degree of deposition of inflammatory cells of the test group and the control group. But the width of the scar of test group was thinner than that of the control group (Refer to FIG. 1). The deposited collagen of Trichrome dyeing was thin and irregular in both the test group and the control group. But the amount of deposition of the collagen of the test group was meager compared to that of the control group, and deposition of the collagen of the control group was larger and denser (Refer to FIG. 2). When the width of scar was measured at magnification of 40, that of the control group was average of 68.44 μm±22.84, and that of the test group was average of 26.66 μm±9.60. As a result of Student t-test, the value of p at a significant level of 0.05 of the two groups was the value of 0.01 or less, which was statistically significant difference (Refer to Table 3).

In the hematoxylin-eosin dyeing of the wound eight (8) weeks after the wound was made, the test group formed a thin and unnoticeable scar and the control group formed a thick and significant scar at the low magnification (×40) (Refer to FIG. 3). At the high magnification (×400), the test group showed thicker and regular collagen fibers which was not much different from the normal dermis nearby, whereas the control group showed compactly deposited collagen fiber, which was clearly distinguishable from the normal dermis nearby. Not many of the fibroblast cells were found in the test group but many of them were still found in the control group. In Trichrome dyeing, the density of collagen fiber in the test group was not different from that of the normal dermis nearby. The fiber was thick and regular, which was recovered close to the dermis, while since thin and irregular fiber was densely deposited, the control group clearly showed the scar (Refer to FIG. 4). When the width of the scar was measured at 40 magnification, that of the control group was average of 37.67 μm±7.98, and that of the test group was average of 13.89 μm±2.42. As a result of Student t-test, the value of p at a significant level of 0.05 of the two groups was the value of 0.01 or less, which was a statistically significant difference (Refer to Table 3 below).

TABLE 3
Comparison of the width of sodium bicarbonate group
and that of control group (measured by using image
analysis program, “Image-Pro version 3.0, Microsoft”)

| | Control Group* | sodium bicarbonate group* | |
|---|---|---|---|
| | (n = 10) | (n = 10) | Value of p |
| 2 Weeks | 68.44 μm ± 22.84 | 26.66 μm ± 9.60 | 0.01≧ |
| 8 Weeks | 37.67 μm ± 7.98 | 13.89 μm ± 2.42 | 0.01≧ |

*Mean ± SD

B. Experiment of TGF-β Inactivation

Two (2) weeks after a wound was made, an inflammation was observed from epidermal tissue layer to the muscular tissue layer at the low magnification (×40) in hematoxylin-eosin dyeing, and there was not much difference between the degree of deposition of inflammatory cells of TGF-β group and sodium bicarbonate+TGF-β group. But the width of the scar of sodium bicarbonate+TGF-β group was thinner than that of the TGF-β group (Refer to FIG. 5). The deposited collagen of Trichrome dyeing was thin and irregular in both the test group and the control group. But the amount of deposition of the collagen of the test group was meager compared to that of the control group, and deposition of the collagen of the control group was larger and denser. When the width of scar is measured at magnification of 40, that of the TGF-β group is average of 74.84 μm±15.93, and that of sodium bicarbonate+TGF-β group was average of 35.41 μm±6.90. As a result of Student t-test, the value of p at a significant level of 0.05 of the two groups was the value of 0.01 or less, which was a statistically significant difference (Refer to Table 4 below).

In the hematoxylin-eosin dyeing of the wound eight (8) weeks after the wound was made, sodium bicarbonate+TGF-β group formed a thinner and less noticeable scar than TGF-β group at the low magnification (×40). The control group formed a thicker and more significant scar than the test group at the low magnification (×40) (Refer to FIG. 6). At the high magnification (×400) as same as the test of alkalinization of a wound, sodium bicarbonate+TGF-β group showed thicker and regular Collagen fibers which was not much different from the normal dermis nearby. In Trichrome dyeing, the density of collagen fiber in sodium bicarbonate+TGF-β group was not that different from the normal dermis tissue nearby. The fiber was thick and regular, which was recovered closed to the dermis tissue, while since thin and irregular fiber was densely deposited, TGF-β group clearly showed the scar (Refer to FIG. 7). When the width of the scar was measured at 40 magnification, that of TGF-β group was average of 72.92 μm±16.04, and that of sodium bicarbonate+TGF-β group was average of 49.09 μm±5.58. As a result of Student t-test, the value of p at a significant level of 0.05 of the two groups was the value of 0.01 or less, which was a statistically significant difference (Refer to Table 4 below).

TABLE 4
Comparison of the width of sodium bicarbonate group
and that of the control group (measured by using image
analysis program, “Image-Pro version 3.0, Microsoft”)

| | Control Group* | sodium bicarbonate group* | |
|---|---|---|---|
| | (n = 10) | (n = 10) | Value of p |
| 2 Weeks | 74.84 μm ± 15.93 | 35.41 μm ± 9.60 | 0.01≧ |
| 8 Weeks | 72.92 μm ± 16.04 | 49.09 μm ± 5.58 | 0.01≧ |

*Mean ± SD

### Example 2

This working example was carried out in accordance with the same manner as described in EXAMPLE 1 but using NaOH or KOH instead of sodium bicarbonate as an alkalinizing agent. This experiment is for the purpose of showing that the reduction of scar formation illustrated in the EXAMPLE 1 resulted from the alkalinization of wound site conditioned by sodium bicarbonate, rather than the activity of the sodium bicarbonate per se.

Materials

Animal: twenty white rats of Spraue-Dawley origin, which weigh 300-350 mg
0.231 mmole NaOH (pH 9.5±0.3)
0.249 mmole KOH (pH 9.5±0.3)
   Methods of Experiment
   A. Experiment of Wound Alkalinization (Ten Animals for NaOH, Ten Animals for KOH)

On the dorsum of adult white rats, skin near the limbs which are on the same distance from the median line was incised in about 10 mm long. After incision, the control group was simply sutured. In test groups, 0.1 mEq NaOH or KOH 0.1 cc (pH 9.5±0.3) was injected to the dermis of the incised wound.
a. control×1 site: suture only
b. experimental×1 sites: 0.1 mEq NaOH 0.1 cc or 0.1 mEq KOH 0.1 cc injection and suture
   Analysis of the Results
   A. Histological Observation

On 14^{th} and 56^{th} days from the experiments, tissues of ten adult white rats (5 animals for NaOH, 5 animals for KOH) were taken, fixed in 10% neutral buffer formalin for 24 hours, embedded in paraffin throughout the normal tissue treating process. In the middle of the wound, the tissue was cut into 4 μm thick, perpendicularly to the major axis, and dyed with hematoxyline-eosine and Masson's trichrome to observe the connective tissues

B. Measurment of the Width of Scar

With a tissue sample, which was cut perpendicularly to the middle of the wound, the width of the scar by use of image analysis program (Image-Pro version 3.0, Microsoft) under 40 magnification was measured, and the differences between the groups were compared. The result of the measurement was confirmed by Student t-test.

Results

The tissues to be tested were obtained from tested twenty white rats, ten of which were sacrificed at each time, after 2 and 8 weeks from the operational handling.

A. Experiment of Wound Alkalinization

Two (2) weeks after a wound was made, an inflammation was observed from the epidermal tissue layer to the muscular tissue layer at the low magnification (×40) in hematoxylin-eosin dyeing, and there was no significant difference between the degree of deposition of inflammatory cells of the test group treated with NaOH and the test group treated with KOH. But the width of the scar of test group was thinner than that of the control group. The test group showed the deposited collagen of Trichrome dyeing thinner than the control group. But the amount of deposition of the collagen of the test group was meager compared to that of the control group, and deposition of the collagen of the control group was larger and denser. These results of the test groups are substantially identical to those of the test group treated with sodium bicarbonate. When the width of scar was measured at magnification of 40, that of the control group was average of 63.20 μm±14.91, and those of the test groups treated with KOH and NaOH were average of 26.50 μm±6.81 and 28.20 μm±5.63, respectively. As a result of Student t-test, the value of p at a significant level of 0.05 of the two groups was the value of 0.01 or less, which was statistically significant difference (see Table 5 below)

Eight (8) weeks after the wound was made, there was no significant histological difference between the test groups treated with KOH and NaOH. The results of the test groups treated with KOH or NaOH were similar to those of the test group treated with sodium bicarbonate. In the hematoxylin-eosin dyeing, at the low magnification (×40), the test groups showed thicker and regular collagen fibers which were not much different from the normal dermis nearby, whereas the control group showed compactly deposited collagen fiber, compared to the test group. In Trichrome dyeing of the test groups, the density of collagen fiber was not different from that of the normal dermis nearby and the fiber was thick and regular, which was recovered closed to the dermis. In contrast, the control group has the thin and irregular fiber densely deposited and clearly showed the scar. When the width of the scar was measured at 40 magnification, that of the control group was average of 30.80 μm±8.91, and those of the test groups treated with KOH and NaOH were average of 14.90 μm±4.35 and 16.80 μm±4.41, respectively. As a result of Student t-test, the value of p at a significant level of 0.05 of the two groups was the value of 0.01 or less, which was a statistically significant difference (see Table 5 below).

TABLE 5
Comparison of the widths of KOH and NaOH groups
and control group (measured by using image analysis
program, “Image-Pro version 3.0, Microsoft”)

| | Control Group* | KOH group* | NaOH group* | Value |
|---|---|---|---|---|
| | (n = 10) | (n = 10) | (n = 10) | of p |
| 2 Weeks | 63.20 μm ± 14.91 | 26.50 μm ± | 28.20 μm ± 5.63 | 0.01≧ |
| | | 6.81 | | |
| 8 Weeks | 30.80 μm ± 8.91 | 14.90 μm ± | 16.80 μm ± 4.41 | 0.01≧ |
| | | 4.35 | | |

*Mean ± SD

Industrial Applicability

As explained and shown in the Examples above, the agent for reducing scar formation according to the present invention by controlling the wound healing process can be clinically and immediately applied to the treatment of tylosis scar and keloid, etc., and is effective to the treatment of intractable fibrosis diseases. Also, the invention can be widely applied in many ways as an easy tool which can control the action of TGF-β in a living organism.

1. A method for reducing scar formation at a wound site comprising administering to said wound site an effective amount of an alkalinizing agent to raise pH at the wound site so as to inactivate TGF-β at the wound site and allowing the wound site to heal thereby reducing scar formation at the healing site.

2. The method according to claim 1, wherein the raised pH at the wound site is in the range of 8.5 to 10.0.

3. The method according to claim 2, wherein the raised pH at the wound site is in the range of 9.2 to 9.8.

4. The method according to claim 1, wherein said alkalinizing agent is injected into dermis tissue of the wound site.

5. The method according to claim 1, wherein said alkalinizing agent is selected from a group consisting of sodium bicarbonate, sodium hydroxide, potassium hydroxide, tromethamine (THAM), acetazolamide (Diamox), sodium citrate, potassium citrate, phosphate salts (potassium phosphate, sodium acid phosphate, and tribasic sodium phosphate), and sodium lactate.

6. The method according to claim 5, wherein said phosphate salt is potassium phosphate, sodium acid phosphate or tribasic sodium phosphate.

7. A pharmaceutical composition for reducing scar formation at a wound site comprising an effective amount of an alkalinizing agent to raise pH at the wound site and inactivate TGF-P at the wound site and a pharmaceutically acceptable carrier.

8. The composition according to claim 7, wherein the raised pH at the wound site is in the range of 8.5 to 10.0.

9. The composition according to claim 8, wherein the raised pH at the wound site is in the range of 9.2 to 9.8.

10. The composition according to claim 7, which is in a form of an injection solution.

11. The composition according to claim 7, wherein said alkalinizing agent is present in a concentration of 0.1 to 1 mEq/0. 1 cc of purified water.

12. The composition according to claims 7, wherein said alkalinizing agent is selected from a group consisting of sodium bicarbonate, sodium hydroxide, potassium hydroxide, tromethamine (THAM), acetazolamide (Diamox), sodium citrate, potassium citrate, phosphate salts (potassium phosphate, sodium acid phosphate, and tribasic sodium phosphate), and sodium lactate.

13. The method according to claim 12, wherein said phosphate salt is potassium phosphate, sodium acid phosphate or tribasic sodium phosphate.

14. A kit for reducing scar formation at a wound site comprising: (i) at least one container containing an effective amount of an alkalinizing agent to raise pH at the wound site and inactivate TGF-β at the wound site and a pharmaceutically acceptable carrier wherein the raised pH at the wound site may be in the range of 8.5 to 10.0; and (ii) written instructions on the use of the kit for reducing scar formation.
